(19) United States
(12) Patent Application Publication
(10) Pub. No.: US 2016/0175177 A1
(43) Pub. Date: Jun. 23, 2016

(54) SYSTEM AND METHOD FOR ADJUSTING THE HEIGHT OF A PATIENT SUPPORT TABLE BASED UPON SENSED PATIENT HEIGHT

(71) Applicant: General Electric Company, Schenectady, NY (US)

(72) Inventors: Sathish Kumar Kambapalli Velayutham, Bangalor (IN); Manjunatha K S, Bangalore (IN); Karthikeyan Sathyamurthy, Bangalore (IN); Munish Vishwas Inamdar, Bangalore (IN); Bhanumathi HR, Bangalore (IN); Sriram Visvanathan Iyer, Bangalore (IN); Deepjyot Kaur, Bangalore (IN)

(73) Assignee: GENERAL ELECTRIC COMPANY, Schenectady, NY (US)

(21) Appl. No.: 14/572,981

(22) Filed: Dec. 17, 2014

(51) Int. Cl. A61G 13/06; G01B 11/02; G01B 17/00; A61B 6/04

(52) U.S. Cl. CPC A61G 13/06; A61B 6/0407; G01B 11/026; G01B 17/00

(57) ABSTRACT

A system and method for automatically adjusting the height of a patient support table based upon a sensed height of a patient. The system includes a patient height sensing device that is positioned to determine the height of a patient. The determined height of the patient is relayed to a controller that calculates a desired patient loading height for the support surface of the table. The controller activates a lift assembly to move the support surface of the support table to the desired patient loading height. Once the patient is positioned on the support table, the controller moves the support, table to a medical procedure height and the medical scanning procedure is carried out. After the procedure is complete, the support table is returned to the desired patient loading height for unloading the patient.

### BACKGROUND

The present disclosure generally relates to a system and method for automatically adjusting the height of a patient support table based upon the height of the patient. More specifically, the present disclosure relates to a system and method that senses the height of a patient and automatically adjusts the height of the patient support table to cater to the needs of a wide variety of patients and increase the ergonomics of the patient support table.

Medical diagnostic imaging and scanning systems, such as magnetic resonance imaging (MRI) apparatus, X-Ray machines, position emission tomography (PET) scanners and computer tomography (CT) scanners are well known in the industry. Such machines are able to carry out medical scanning procedures that are able to diagnose various medical conditions, such as internal injuries, cancerous tumors and the like.

In order to obtain an image of a patient with the different types of CT and MRI scanners, it is necessary that the patient be located exactly at a predetermined position within the scanning device. For such reason, the scanning devices include a patient support table that is movable vertically to properly position the patient within the scanner. In addition, the patient support table is movable horizontally to move the patient into and out of the scanning device.

Although such patient support tables are known, the height of the patient support table typically must be manually adjusted by medical personnel in the testing room. Such adjustment requires the medical personnel to manually operate controls to lower the patient support table to a desired position that allows the patient to be loaded onto the table. Such adjustment requires additional time and effort by the medical personnel in the testing room. Further, if such adjustments are not carried out, loading the patient onto the patient support table will either decrease the comfort of the patient or require the medical personnel to physically assist the patient onto the patient support table.

### SUMMARY

The present disclosure generally relates to a system and method for automatically adjusting the height of a patient support table based upon the sensed height of a patient. The automatic adjustment of the height of the patient support table to a desired patient loading height aids in the ingress and egress ala patient relative to a support surface of the patient support table.

The system of the present disclosure includes a patient support table that supports a patient during the performance of a medical procedure, such as a medical scanning procedure. The support table includes a support platform that is configured to receive the patient. The support platform is coupled to a lift assembly that is operable to adjust the height of the support platform from a floor. The lift assembly can include an actuator that is automatically controlled to raise and lower the support platform.

The system further includes a sensing device that is positioned to detect the height of a patient. In one embodiment of the disclosure, the sensing device is positioned to detect the height of the patient as the patient passes beneath a sensor. Such sensor could be an ultrasound sensor or a laser sensor.

In a contemplated alternate embodiment, the sensing device could be a camera mounted to capture an image of the patient. Based upon the known location of the camera, the image from the camera is used to determine the height of the patient.

In yet another alternate embodiment, the sensing device is an LED array that includes a plurality of spaced LED emitters and a plurality of spaced receivers. When the patient passes through the LED array, the sensing device is able to determine the height of a patient.

The determined height of the patient is relayed to a controller. The controller is coupled to the lift assembly such that the controller can control the raising and lowering of the support platform through operation of the lift assembly. When the controller receives the detected height of the patient, the controller calculates a desired patient loading height. The desired patient loading height is a height selected to aid in the ingress and egress of a patient from the support platform of the patient support table.

The controller operates the lift assembly to move the support platform to the desired patient loading height. In one embodiment of the disclosure, the system further includes a platform height detector that is positioned to determine the actual height of the platform from the floor. In one embodiment, the platform height detector is an encoder that can determine the actual height of the support platform from the floor.

As the height of the support platform is adjusted, the controller monitors the actual height of the platform relative to the desired patient loading height. The controller controls operation of the lift assembly until the support platform reaches the desired patient loading height.

After the patient has been loaded onto the support platform, the system and method of the disclosure raises the support platform to a medical procedure height. The medical procedure height is the desired height of the support table for carrying out the medical procedure.

Once the medical procedure has been completed, the system and method returns the support platform to the desired patient loading height. Once the support platform has returned to the desired patient loading height, the patient can be removed from the support platform.

Various other features, objects and advantages of the invention will be made apparent from the following description taken together with the drawings.

### BRIEF DESCRIPTION OF THE DRAWINGS

The drawings illustrate the best mode presently contemplated of carrying out the disclosure. In the drawings:

FIG. 1 is a top end perspective view showing a patient support table positioned at a medical procedure height in front of a medical scanning device;

FIG. 2 is a side elevation view of the lift assembly positioning the support platform at a medical procedure height;

FIG. 3 is a side view similar to FIG. 2 showing the support platform at a lowered, patient loading height;

FIG. 4 is a schematic illustration of a first embodiment of a sensing device for determining the height of a patient as the patient enters the testing room;

FIG. 5 illustrates a second embodiment of the sensing, device for determining the height of the patient as the patient enters the testing room;

FIG. 6 illustrates a third embodiment of the sensing device used to determine the height of the patient as the patient enters the testing room;

FIG. 7 is a schematic illustration of the operational components of the system of the present disclosure; and

FIG. 8 is a flow chart showing the steps carried out by the system of the present disclosure.

### DETAILED DESCRIPTION

In the present description, certain terms have been used for brevity, clearness and understanding. No unnecessary limitations are to be applied therefrom beyond the requirement of the prior art because such terms are used for descriptive purposes only and are intended to be broadly construed. The different systems and methods described herein may be used alone or in combination with other systems and methods. Various equivalents, alternatives and modifications are possible within the scope of the appended claims. Each limitation in the appended claims is intended to invoke interpretation under 35 U.S.C. §112, sixth paragraph, only if the terms “means for” or “step for” are explicitly recited in the respective limitation.

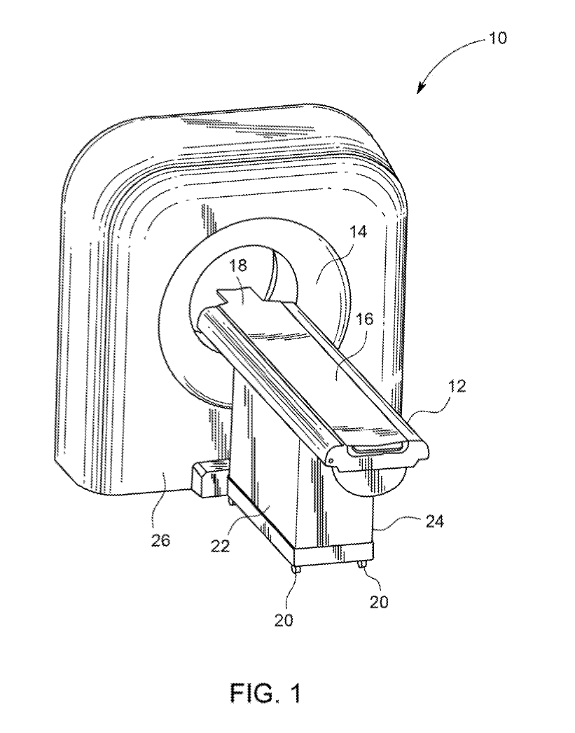

FIG. 1 it lustrates a medical scanning device 10 that is shown with an exemplary embodiment of a patient support table 12 constructed in accordance with the present disclosure. As is known to one skilled in the art, the scanning device 10 includes an annular gantry 14 containing a field generator that projects a beam of rays toward a detector array on the opposite side of the gantry. The scanning device 10 is capable of carrying out a medical scanning procedure on a patient when the patient is supported on a support platform 16 of the support table 12. The support platform 16 includes a movable section 18 that can be extended such that the patient is moved through the annular gantry 14.

In the embodiment shown in FIG. 1, the patient support table 12 is supported on a series of wheels 20 such that the patient support table 12 can be moved toward and away from the scanning device 10. However, the patient support table 12 could be stationary while operating within the scope of the present disclosure. The support platform 16 is mounted to a base 22 which includes a lift assembly (not shown) concealed within an outer shroud 24. The lift assembly contained within the base 22 allows the height of the support platform 16 to be adjusted relative to the floor 26. The height of the support platform 16 relative to the floor 26 can be adjusted to both aid in the loading of a patient onto the support platform 16 and to present the patient at the correct height for introduction into the annular gantry 14.

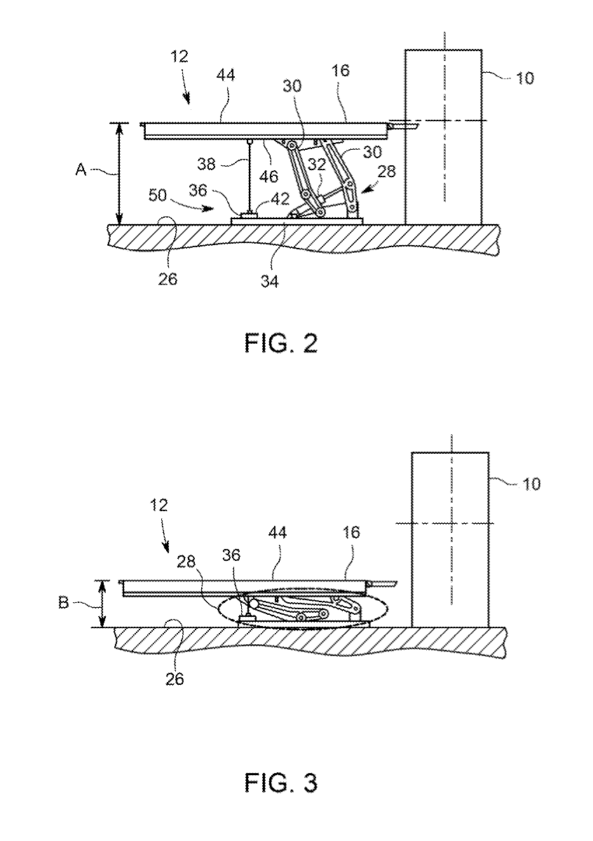

FIGS. 2 and 3 illustrate one embodiment of a lift assembly 28 that can be used to adjust the height of the support platform 16 relative to the floor 26. In the embodiment illustrated, the lift assembly 28 includes a pair of support arms 30 connected to a table actuator 32. The table actuator 32 is securely attached to a lower support 34. The table actuator 32 can be selectively activated to move the support platform 16 from an elevated medical procedure height A shown in FIG. 2 to a lowered patient loading height B shown in FIG. 3. The position of the support platform 16 can be modified between upper and lowermost positions such that the support table 12 can be utilized with different scanning devices 10 and with patients having different sizes.

In the embodiment illustrated in FIGS. 2 and 3, the table actuator 32 is shown as a cylinder having a rod that can be extended and retracted to raise and lower the support platform 16. However, it should be understood that various different types of lift assemblies 28 could be utilized while operating within the scope of the present disclosure, As an example, the lift assembly could include a series of scissor arms and the table actuator 32 could be an electronic motor that operates to adjust the position of the scissor arms to raise and lower the support platform 16. It is contemplated that these various different lift assemblies and table actuators could be utilized while operating within the scope of the present disclosure.

In the embodiment shown in FIGS. 2 and 3, the patient support table 12 includes a platform height detector 50, such as an encoder 36, that is positioned to determine the height of the support platform 16 from the floor 26. In the embodiment shown, the encoder 36 is a string encoder that includes a retractable cable 38 attached to the bottom surface 40 of the support platform 16. As the support platform moves upward and downwardly, the cable 38 is retracted into and pulled from a body 42 which is mounted to the support base 34. The extension and retraction of the cable 38 is sensed by an internal element of the encoder 36 such that the encoder 36 can determine the actual height of the top support surface 44 of the support platform 16 from the floor 26.

Referring now to FIG. 7, the control and operating system 46 of the present disclosure includes a microprocessor based controller 48. The microprocessor based controller 48 can be mounted within the base 22 of the patient support table 12 and controls the operation of the system of the present disclosure. As shown in FIG. 7, the controller 48 is in communication with the platform height detector 50. In the embodiment shown in FIGS. 2 and 3, the platform height detector 50 is the string encoder 36. However, it is contemplated that other devices could function as the platform height detector while operating within the scope of the present disclosure. For example, any type of device that is able to sense the distance from the floor 26 to the support platform 16 could be utilized while operating within the scope of the present disclosure.

Referring back to FIG. 7, the controller 48 is in communication with the actuator of the lift assembly, as shown by block 52. In the embodiment shown in FIG. 7, the controller 48 is in direct communication with the lift assembly actuator 52. However, it is contemplated that an additional controller could be positioned between the main controller 48 and the lift assembly actuator 52. In such an embodiment, the controller 48 would control the actuation of the lift assembly through the intermediate controller. However, it should be clear from FIG. 7 that the controller 48 is able to control the actuation of the lift assembly actuator to raise and lower the support platform.

As indicated in FIG. 7, the controller 48 is in further communication with a patient height sensing device 54, The patient height sensing device 54 operates to detect the height of a patient before the patient is positioned on the support platform. The height of the patient is relayed to the controller 48. Based upon the detected height of the patient, the controller 48 determines a desired loading height for the support platform and controls the lift assembly actuator 52 to adjust the height of the support platform from the floor.

In the embodiment of the present disclosure, as shown in FIGS. 2 and 3, the support platform 16 is movable between the medical procedure height A shown in FIG. 2 and the desired patient loading height B shown in FIG. 3. In the embodiment shown, it is contemplated that the height of the support platform 16 can be adjusted between 400-970 mm. In this manner, the support platform 16 can be moved to the desired patient loading height B shown in FIG. 3 to allow a patient to be loaded onto the support surface 44. By adjusting the desired patient loading height B, the system of the present disclosure is able to position the support platform 16 to accommodate patients having different height and to create a comfortable, ergonomic ingress or egress for the patient both prior to and after a medical scanning procedure is performed.

The controller 48 shown in FIG. 7 receives the sensed patient height from the patient height sensing device 54 and calculates a desired patient loading height for the patient. The desired patient loading height is determined directly based upon the sensed patient height such that the patient support table 12 can more easily be used with patients having a variety of different heights. In addition, if the controller 48 determines that the patient is in a wheelchair, the controller 48 can actuate the lift assembly actuator 52 and lower the support platform to its lowest possible position to further aid in loading the immobilized patient onto the support platform.

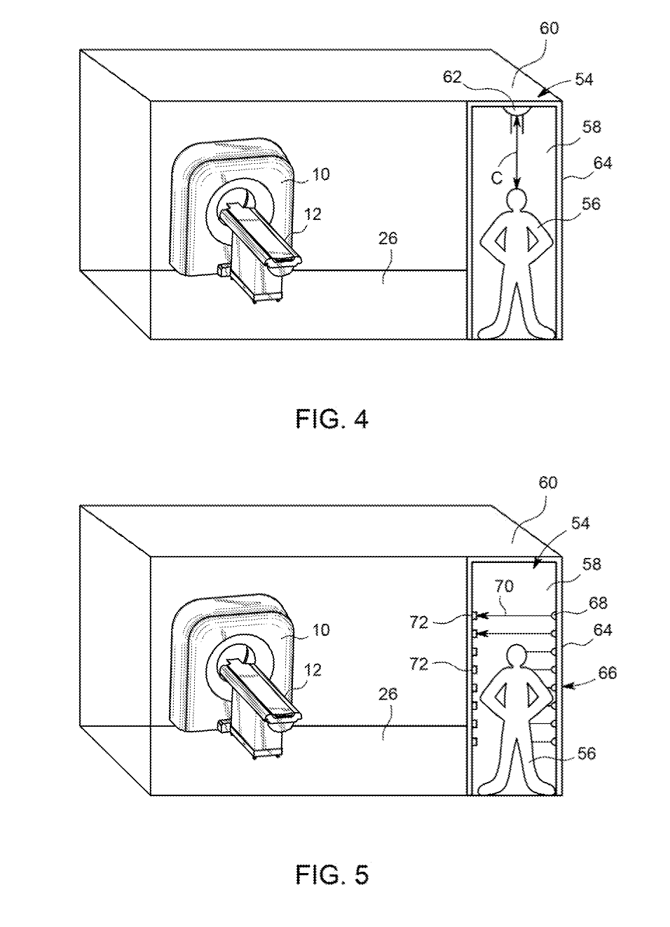

FIG. 4 illustrates a first embodiment of the patient height sensing device 54. In the embodiment shown in FIG. 4, a patient 56 enters the testing room 60 through a doorway 58. The testing room 60 includes the support table 12 and the scanning device 10.

In the embodiment of FIG. 4, the patient height sensing, device 54 includes an overhead sensor 62. In the embodiment shown, the sensor 62 is mounted to the top of the door frame 64 or the ceiling of the testing room 60 such that the patient 54 passes beneath the overhead sensor 62 upon entering into the testing room 60. However, it should be understood that the sensor 62 could be mounted at other locations as long as the sensor 62 is able to detect the distance C between the sensor 62 and the patient 56. The detected distance C is used along with a known distance from the sensor 62 to the floor 26 to estimate the height of the patient 56. In one embodiment of the disclosure, the doorway 58 can include patient indicators on the floor to indicate to the patient 56 where the patient should stand to be located beneath the overhead sensor 62.

In one embodiment of the disclosure, the overhead sensor 62 is an ultrasound sensor. In another, alternate embodiment, the overhead sensor 62 can be a laser sensor. In each case, the overhead sensor 62 determines the distance C and, based upon this detected distance, calculates the height of the patient 56 based upon the difference between the detected distance C and the distance from the sensor 62 to the floor 26.

Once the sensor 62 determines the height of the patient, the detected height is relayed to the controller 48, as is shown in FIG. 7.

FIG. 5 illustrates a second embodiment of the patient height sensing device 54. In the second embodiment, the patient height sensing device 54 includes an LED array 66 that is positioned on opposite sides of the door frame 64 to detect the height of the patient 56 as the patient passes through the doorway 58. As illustrated in FIG. 4, the LED array 66 includes a series of spaced LED emitters 68 that each emit a beam of light 70 that is received by an opposite receptor 72. The emitters 68 and receptors 72 are aligned with each other such that the light 70 from each emitter 68 is received by one of the receptors 72.

As can be seen in FIG. 5, when the patient 56 passes through the doorway 58, the patient 56 blocks a number of light beams. The LED array 66 determines the height of the patient 56 based upon the number of receptors 72 that detect light beams. In this mariner, the LED array 66 is able to detect the height of the patient and relay this height information to the controller 48.

As illustrated in FIG. 5, the LED array 66 includes a number of LED emitters 68 that are positioned relatively low along the doorway 58, The lowest LED emitters 68 are able to detect when a patient enters the room in a wheelchair. If the patient is seated in a wheelchair, the controller will activate the lift assembly actuator to bring the support platform of the support table to its lowest position. In one contemplated embodiment of the disclosure, if a patient is brought into the testing room 60 in a wheelchair, the operator can signal that the patient is in a wheelchair, which causes the controller 48 to sense a “patient sitting height”.

In yet another alternate embodiment, if the patient is brought into the testing room 60 on a stretcher, the LED array 66 can detect the height of the stretcher and the controller adjusts the height of the support platform to closely correspond to the height of the stretcher to further facilitate an easy transfer of the patient from the stretcher to the patient support table.

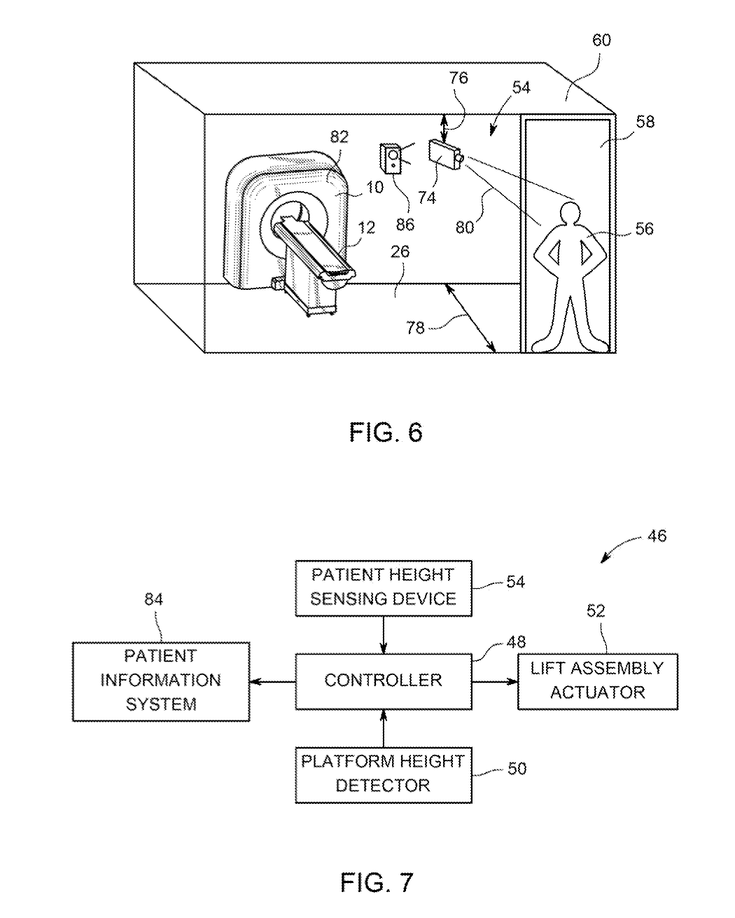

FIG. 6 illustrates yet another alternate embodiment for the patient height sensing device 54. In the embodiment shown in FIG. 6, the patient height sensing device 54 includes a camera 74 mounted within the testing room 60. In the embodiment shown in FIG. 6, the camera 74 is mounted to a back wall of the room 60 having a known distance 76 from the ceiling and a known distance 78 from the entrance to the doorway 58. The camera 74 has a field of view 80 that is able to capture an image of the patient 56 as the patient enters the test room 60 through the doorway 58. The digital image recorded by the camera can he interpreted along with the distances 76 and 78 to determine the height of the patient 56. The height of the patient 56 is relayed to the controller 48 as in the previous embodiments.

It is contemplated that in one embodiment of the camera based system shown in FIG. 6, the patient 56 could be made to wear a gown or other distinguishing clothing that will aid in image processing to differentiate the patient from a doctor/radiologist who may also enter into the testing room 60 through the doorway 58. Alternatively, the operator could be prompted to signal when the patient 56 is entering into the room such that the recorded image would be only of the patient.

Although the camera 74 is shown mounted to one of the walls, it is contemplated that the camera could be mounted at other locations, such as on the front face 82 of the scanning device 10. In each instance, the image, along with data related to the distance of the camera from the door and the height of the camera above the floor are relayed to the controller such that the controller can make calculations to determine the height of the patient 56.

As can be understood in the alternate embodiments of FIGS. 4-6, the patient height sensing device 54 is operable to determine the height of the patient as the patient passes through the doorway 58 prior to loading of the patient on the support table. Alternatively, the patient height sensing device 54 can be located at other locations throughout the testing, room while operating, within the scope of the present disclosure.

Referring back to FIG. 7, the system 46 can also include a patient information system 84. The patient information system 84 allows the controller 48 to communicate with the patient as the patient is within the testing room and is having the medical procedure carried out, in one embodiment of the disclosure, the patient information system can include a speaker 86 mounted within the test room, as illustrated in FIG. 6. The speaker 86 is able to communicate audible messages to the patient as the medical procedure is carried out utilizing the scanning device 10. The patient information system 84, including the speaker 86, allows the system to fill the gap between operator instructions and provides consistent and dependable information to the patient to avoid ambiguity of actions when the patient is having the medical procedure performed for the first time.

In one embodiment of the disclosure, the patient information system can initially inform the patient that the patient should make sure that the patient does not have any medical implants, jewelry or makeup and to prompt the user to move to a designated area for height measurement. The patient information system then advises the patient that the patient support table will move to a comfortable sitting height and will then prompt the patient to lie down on the patient support table. Once the patient is in position, the patient information system 84 will advise the patient that the table will move to a medical procedure height and that the scanning will take place over a predetermined period of time, such as 30-40 minutes.

Once the medical scanning procedure has been completed, the patient information system will advise the patient that the table will move down to the comfortable patient loading height that will allow the patient to be removed from the patient support table.

As can be understood by the above description and FIGS. 6 and 7, the patient information system 84 further facilitates the medical procedure and eliminates steps that would otherwise need to be carried out by the operator.

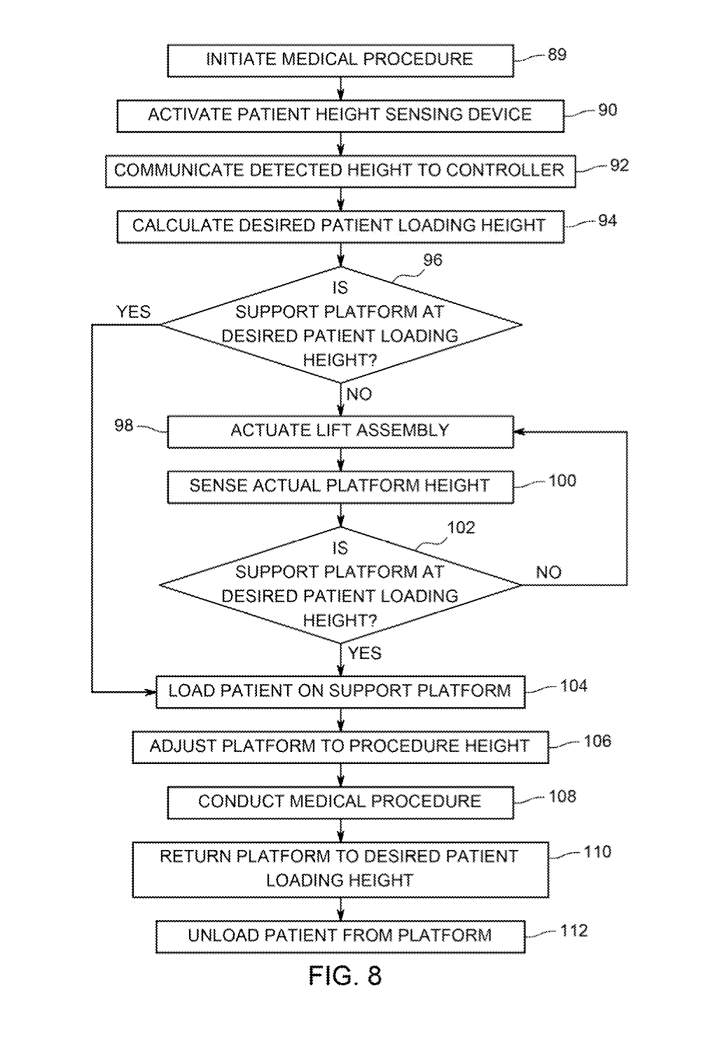

FIG. 8 illustrates the steps of the method carried out in accordance with the present disclosure. The flowchart of FIG. 8 is meant to illustrate only one embodiment of the disclosure and is not meant to be limiting as to the steps that may be performed by the system of the present disclosure. Initially, the medical procedure is initiated in step 88. Initiation of the medical procedure can be done through a manual activation by an operator or could be initiated by the sensing of the patient entering into the room.

Once the medical procedure has been initiated, the system activates the patient height sensing device as shown in step 90. In the embodiment shown in FIGS. 4-6, the patient height sensing device 54 detects the height of the patient 56 as the patient passes through the doorway 58. The three embodiments shown in FIGS. 4-6 are each operable to determine the height of the patient and this detected height is communicated to the controller 48, as shown in step 92. The communication from the patient height sensing device 54 to the controller 48 allows the controller 48 to learn the height of the patient that will undergo the medical procedure. Once the controller 48 receives the detected height of the patient, the controller calculates a desired patient loading height in step 94. As indicated previously, the controller calculates the desired patient loading height based upon the sensed height of the patient and whether the patient is either in a wheelchair or on a stretcher.

Once the desired patient loading height is determined, the system moves to step 96 in which the system determines whether the support platform is currently at the desired patient loading height. Typically, the support platform of the support table will not be at the desired patient loading height and the system will move to step 98. In step 98, the system actuates the lift mechanism to move the support platform toward the desired patient loading height. As the lift assembly is actuated in step 98, the system also senses the actual platform height in step 100. The actual platform height is determined by the platform height detector 50, which is shown by the encoder 36 in FIGS. 2 and 3.

In step 102, the controller determines whether the actual height of the support platform is the same as the desired patient loading height. If the actual height of the platform is not the same as the desired loading height, the system returns to step 98 and continues to actuate the lift assembly. This process continues until the controller determines in step 102 that the actual platform height is the same as the desired patient loading height. Once this determination is made in step 102, the system moves to step 104 and indicates that the patient should be positioned on the support platform. In the embodiment that includes the patient information system, the controller can generate an audible message to the patient that the patient can now lay on the support platform.

Once the patient has been positioned on the support platform, the controller generates a signal to the lift assembly to adjust the platform height to a medical procedure height, as shown in step 106. As previously described, the support platform is moved to the medical procedure height such that the support table can be positioned adjacent to the scanning device as shown in FIG. 1 and the patient can be moved into the scanning device. The medical procedure height is typically a known value based upon the size of the scanning device and the support table.

Once the patient has been positioned within the scanning device, the medical procedure is carried out in step 108. The controller may generate an audible message through the patient information system to indicate to the patient that the medical procedure is being conducted.

After the medical procedure has been completed, the system moves to step 110, at which time the support platform is returned to the desired patient loading height. Since the desired patient loading height is specific for the patient to aid the patient in initially sitting on the support platform, the desired patient loading height is also utilized to allow the patient to easily get off of the support platform, as indicated in step 112.

As can be understood by the method and system described by the flowchart of FIG. 8, the system provides for the comfortable ingress/egress of patients from the patient support table and reduces the overall setup time within the testing room. The voice commands generated by the patient information system provides for a patient-friendly environment and the automated workflow helps fill the gaps in the instructions provided by the operator.

This written description uses examples to disclose the invention, including the best mode, and also to enable any person skilled in the art to make and use the invention. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

We claim:

1. A system for adjusting, the height of a patient support table that supports a patient during a medical procedure, comprising:
a support platform for receiving the patient;
a lift assembly coupled to the support platform and operable to adjust a platform height of the support platform from a floor;
a sensing device positioned to detect a patient height; and
a controller positioned to receive the detected patient height, wherein the controller is in operative communication with the lift assembly such that the controller controls the lift assembly to adjust the height of the support platform based upon the detected height of the patient.

2. The system of claim 11 wherein the sensing device is positioned to detect the height of the patient prior to the patient being positioned on the support platform.

3. The system of claim 1 further comprising a platform height detector positioned to determine a platform height of the support platform from the floor, wherein the controller is in communication with the platform height detector.

4. The system of claim 3 wherein the controller receives the determined platform height and actuates the lift assembly until the support platform reaches a desired procedure height, wherein the desired procedure height is determined by the controller based upon the detected patient height.

5. The system of claim 1 wherein the sensing device is a sensor position to determine the patient height as the patient passes beneath the sensor.

6. The system of claim 5 wherein the sensor is an ultrasound sensor.

7. The system of claim 5 wherein the sensor is a laser sensor.

8. The system of claim 1 wherein the sensing device is a camera mounted to capture an image of the patient.

9. The system of claim 1 wherein the sensing device is an LED array including a plurality of spaced LED emitters and a plurality of spaced receivers.

10. A method of automatically adjusting the height of a patient support table, comprising:
positioning a sensing device in a location to sense the patient prior to the patient being supported on the patient support table;
determining the height of the patient based upon information from the sensing device;
communicating the determined height of the patient to a controller; and
utilizing, the controller to operate a lift assembly of the patient support table to position a support platform at a desired patient loading height based upon the determined patient height.

11. The method of claim 10 wherein the controller calculates the desired patient loading height based upon the determined height of the patient.

12. The method of claim 10 further comprising the steps of
determining an actual height of the support platform from a floor as the lift assembly is operated; and
communicating the determined actual height of the support platform to the controller.

13. The method of claim 10 wherein the sensing device is positioned to determine the height of the patient as the patient passes beneath the sensing device.

14. The method of claim 13 wherein the sensing device is an ultrasound sensor.

15. The method of claim 13 wherein the sensing device is a laser sensor.

16. The method of claim 10 wherein the sensing device is a camera mounted to capture an image of the patient.

17. The method of claim 10 wherein the sensing device is an LED array including a plurality of spaced LED emitters and a plurality of spaced receivers.

18. The method of claim 10 further comprising the steps of:
utilizing, the controller to operate the lift assembly to position the support platform at a medical procedure height after the patient is positioned on the support platform for carrying out the medical procedure; and
utilizing the controller to operate the lift assembly to position the support platform at the desired procedure height for removing the patient from the support platform after the completion of the medical procedure.

19. An adjustable patient support table for supporting a patient during the performance of a medical scanning procedure, comprising:
a support platform for receiving the patient and supporting the patient during, the performance of the medical scanning procedure;
a lift assembly coupled to the support platform and operable to adjust the height of the support platform from a floor;
a sensing device positioned to detect a height of the patient prior to the patient being positioned on the patient support table;
a controller positioned to receive the detected height of the patient and in operative communication with the lift, assembly, wherein the controller determines a desired loading height for the support platform based upon the detected height of the patient; and
a platform height detector operable to determine an actual platform height of the support platform from the floor, wherein the controller controls the operation of the lift assembly to position the platform at the desired procedure height based upon the actual platform height received from the platform height detector.

20. The patient support table of claim 19 further comprising a patient information system operable to audibly communicate with the patient during the medical sensing procedure.
